SEQUENCE LISTING (1 sequence)

SEQ ID NO: 1 - DNA, 11 bp, Homo sapiens
gcctccctcct

(19) United States
(12) Patent Application Publication
(10) Pub. No.: US 2015/0252415 A1
(43) Pub. Date: Sep. 10, 2015

(54) ARID1B AND NEUROBLASTOMA

(71) Applicants: THE JOHNS HOPKINS UNIVERSITY, Baltimore, MD (US); THE CHILDREN'S HOSPITAL OF PHILADELPHIA, Philadelphia, PA (US)

(72) Inventors: Bert Vogelstein, Baltimore, MD (US); Kenneth W. Kinzler, Bel Air, MD (US); Victor Velculescu, Dayton, MD (US); Luis Diaz, Ellicot City, MD (US); Nickolas Papadopoulos, Towson, MD (US); Mark Sausen, Baltimore, MD (US); Rebecca Leary, Baltimore, MD (US); John Maris, Philadelphia, PA (US); Michael Hogarty, Philadelphia, PA (US)

(73) Assignees: THE CHILDREN'S HOSPITAL OF PHILADELPHIA, Philadelphia, PA (US); THE JOHNS HOPKINS UNIVERSITY, Baltimore, MD (US)

(21) Appl. No.: 14/435,998

(22) PCT Filed: Oct. 14, 2013
(86) PCT No.: PCT/US2013/064838

§ 371 Date: Apr. 15, 2015

Related U.S. Application Data
(60) Provisional application No. 61/713,875, filed on Oct. 15, 2012.

(51) Int. Cl. C12Q 1/68; G01N 33/50

(52) U.S. Cl. CPC C12Q 1/6874; G01N 33/5011; C12Q 1/686; G01N 2500/10; A61K 48/00

(57) ABSTRACT

Neuroblastomas are tumors of peripheral sympathetic neurons and are the most common solid tumor in children. We performed whole-genome sequencing (6 cases), exome sequencing (16 cases), genome-wide rearrangement analyses (32 cases), and targeted analyses of specific genomic loci (40 cases) using massively parallel sequencing to determine the genetic basis for neuroblastoma. On average, each tumor had 19 somatic alterations in coding genes (range, 3-70). Chromosomal deletions and sequence alterations of chromatin remodeling genes, ARID1A and ARID1B, were identified in 8 of 71 neuroblastomas (11%), and these were associated with early treatment failure and decreased survival. These results highlight dysregulation of chromatin remodeling in pediatric tumorigenesis and provide new approaches for the management of neuroblastoma patients.

### TECHNICAL FIELD OF THE INVENTION

This invention is related to the area of cancer. In particular, it relates to neuroblastoma.

### BACKGROUND OF THE INVENTION

Neuroblastomas are pediatric tumors arising from neural crest-derived precursors of the peripheral sympathetic nervous system. As is typical of embryonal tumors, they arise early in childhood with 90% of all cases diagnosed before the age of 5 years. They are the most common extra-cranial solid tumor of childhood and are responsible for up to 15% of childhood cancer-related deaths¹⁻³, with the majority of patients presenting with metastatic disease at the time of diagnosis. Neuroblastomas manifest marked heterogeneity in clinical outcome. The prognosis of children less than 18 months old, even those with metastatic disease, is favorable, and the tumors in children with stage 4S disease frequently regress spontaneously⁴. Unfortunately, children older than 18 months old who are diagnosed with advanced stage disease have a grave prognosis despite multimodal, dose-intensive chemoradiotherapy⁵. Several recurrent genetic alterations have been elucidated, including amplification of the MYCN oncogene in ˜20% of cases^{6,7}, activating mutations in the ALK tyrosine kinase in ˜8% of primary tumors⁸⁻¹¹, and more recently mutations in ATRX in neuroblastomas presenting in older children and adolescents¹². MYCN amplification is associated with advanced tumors and poor outcome, ATRX mutations define indolent neuroblastoma with eventual progression, while the prognostic value of ALK alterations remains to be defined⁷.

There is a continuing need in the art to improve the diagnosis, prognosis, and treatment of neuroblastomas.

### SUMMARY OF THE INVENTION

According to one aspect of the invention a method detects neuroblastoma in an individual who has or is suspected of having neuroblastoma. A biological sample of an individual is tested to detect a deletion or mutation in ARID1B. The presence of a neuroblastoma in the individual is identified if the deletion or mutation is detected. Identification of the deletion or mutation indicates decreased overall survival risk or presence of minimal residual disease after potentially curative therapy; or the level of ARID1B with the deletion or mutation in the biological sample is a biomarker of response to therapy.

According to another aspect of the invention a method is provided for categorizing a neuroblastoma. Tissue, cells, or shed nucleic acids of a neuroblastoma are tested for a deletion or mutation in ARID1B. The neuroblastoma is assigned to a set based on the presence of the deletion or mutation. The set may be used for predicting outcome, assigning to a clinical trial group, monitoring, or prescribing a therapy, for example.

According to another aspect of the invention a method of inhibiting growth of neuroblastoma cells is provided. A polynucleotide encoding a wild-type ARID1B protein is administered to neuroblastoma cells. The growth of the neuroblastoma cells is thereby inhibited.

Another aspect of the invention is a method to generate a model of neuroblastoma. A mutation is introduced into at least one ARID1B allele in a cell, thereby forming a model of neuroblastoma.

Another aspect of the invention is a method for testing candidate therapeutic agents for treating neuroblastoma. A candidate therapeutic agent is contacted with a cell comprising at least one ARID1B allele that is mutant or deleted. The effect of the agent on growth of the cell is observed. An agent which reduces the growth rate of the cell is a more likely candidate therapeutic agent than one that does not.

Yet another aspect of the invention is a method of testing candidate therapeutic agents for treating neuroblastoma. An ARID1B protein is contacted with an inhibitor. The ARID1B protein is contacted with a candidate therapeutic agent. A candidate therapeutic agent is identified as a more likely candidate therapeutic agent if the agent relieves the inhibition caused by the inhibitor.

These and other embodiments which will be apparent to those of skill in the art upon reading the specification provide the art with

### BRIEF DESCRIPTION OF THE DRAWINGS

FIG. 1. Number and type of somatic alterations detected in each neuroblastoma case. The vertical axis includes non-synonymous single base substitutions, insertions, deletions, and splice site changes (NS Mutations), homozygous deletions and amplifications affecting protein encoding genes, and rearrangements with at least one breakpoint within the coding region of a gene. The inset shows the mutation spectra of somatic non-silent single nucleotide mutations in 16 cases of neuroblastoma. Data on rearrangements and copy number changes were not available for starred samples.

FIG. 2 Genomic alterations in ARID1A and ARID1B. The schematic represents the ARID1B and ARID1A proteins with the predicted effects of observed intragenic deletions and point mutations.

FIG. 3. Overall survival according to ARID1 status. The hazard ratio for death among patients with wildtype ARID1B/A (n=48), as compared to those with mutant ARID1B/A (n=7) was 4.49 (95% confidence interval, CI 1.24-16.33; P=0.0226, log-rank test). The median survival was 1689 days for patients with wildtype ARID1B/A compared to 386 days for patients with mutated ARID1B/A. An analysis that also included hemizygous deletions of the entire coding region of ARIDB further increased the significance of the survival difference between patients with mutant and wildtype ARID1B/A (hazard ratio, HR 6.41; 95% confidence interval, CI 1.93-21.25; P=0.0024, log-rank test).

FIG. 4. Summary of next generation sequencing analyses in neuroblastoma. In total, 16 neuroblastomas were analyzed by whole-exome sequencing, 6 of which were also analyzed by high-coverage whole-genome sequencing; 32 neuroblastomas were analyzed by low-coverage whole-genome sequencing (including 7 with exome sequencing); and 40 independent neuroblastomas were examined by massively parallel sequencing of captured DNA enriched for the MYCN, ALK, ARID1A and ARID1B loci. The total number of tumors analyzed is 74 as two companion cell lines from the same individual at different time-points of therapy were used in the targeted capture analyses.

FIG. 5. CIRCOS plots depicting the genomic landscape of 13 neuroblastoma tumors. The outer ring consists of a chromosomal karyotype with copy number alterations in the inner ring (red) and sequence alterations between the concentric circles (blue). Genomic rearrangements are shown as arcs (green) that span two loci. Genes symbols of recurrent alterations affected by tumor-specific point mutation, rearrangement, or focal copy number changes are indicated adjacent to each plot (specific alterations are listed in Table 2 and Supplementary Tables 5, 6 and 7).

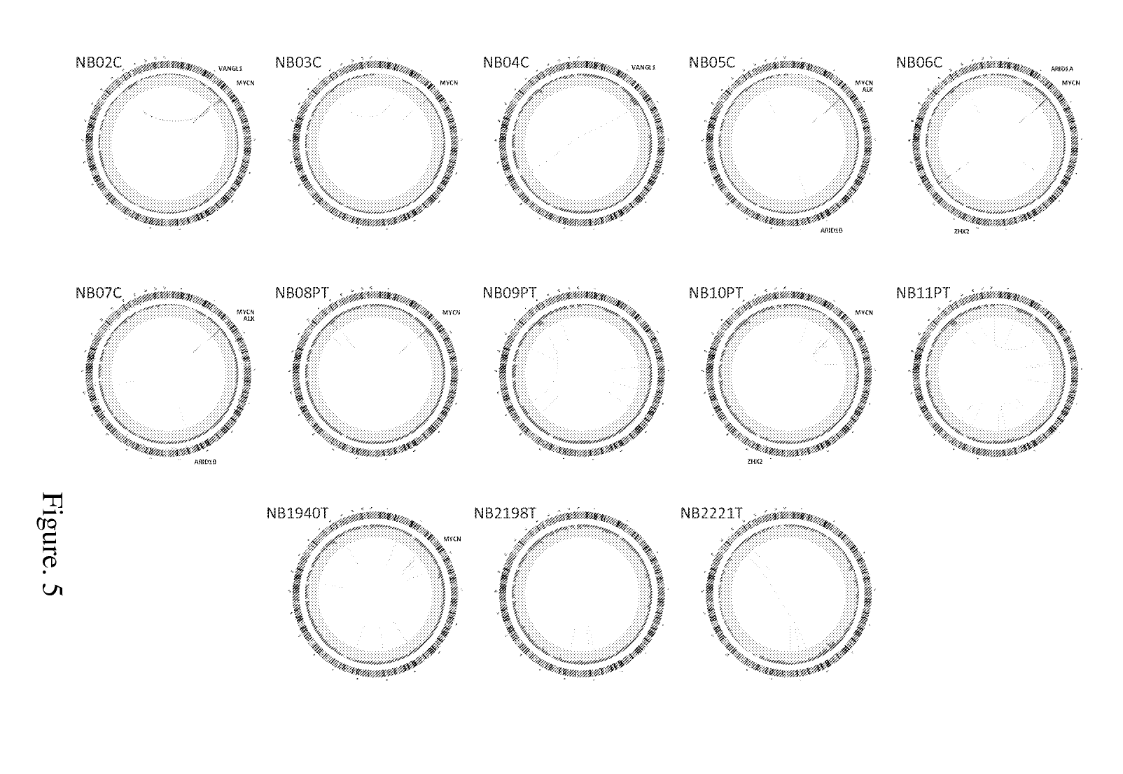

FIG. 6. Detection of minimal residual disease in the circulation of neuroblastoma patients. The presence of circulating tumor DNA in the plasma of four neuroblastoma cases was assessed using tumor-specific rearrangement biomarkers after standard high-risk therapy and during a minimal residual disease immunotherapy trial. Each patient underwent chemo-radiotherapy, autologous stem cell transplantation and surgery prior to the initiation of the ANBL0032 trial³⁹. Plasma (1-2 mL) was collected at the indicated time points prior to and during immunotherapy.

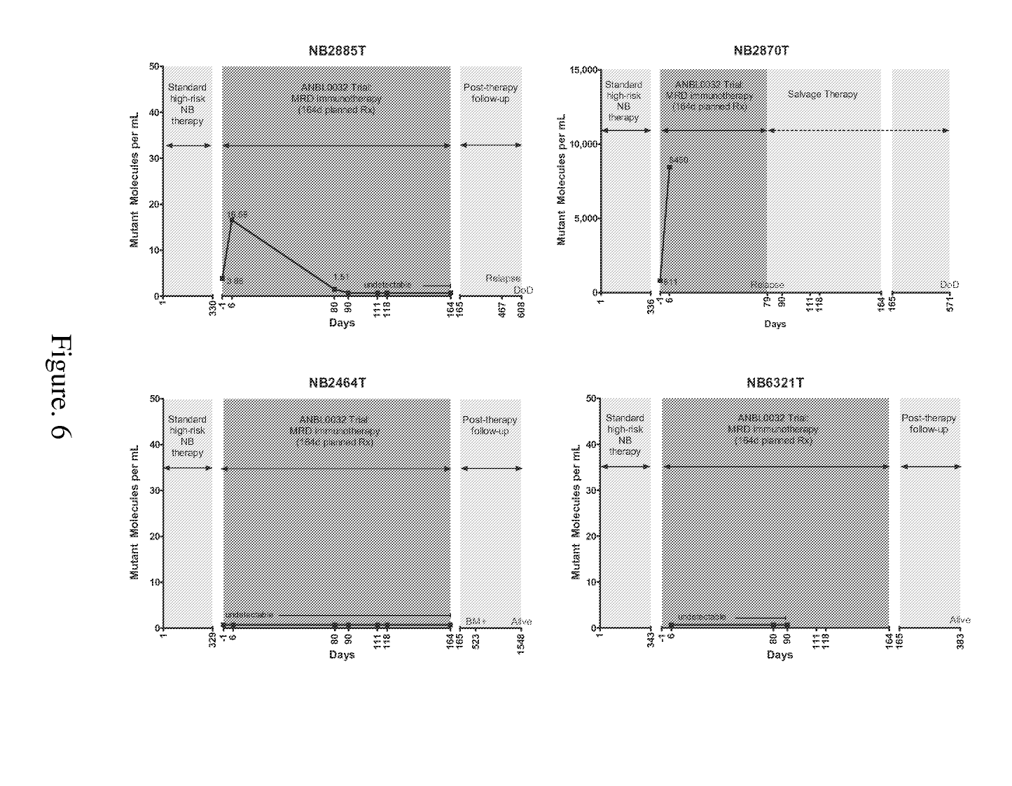

FIG. 7. mRNA expression of npBAF, nBAF and neuritogenic target genes across neuroblastoma risk groups. Transcriptome profiles of n=101 primary neuroblastomas using the Affymetrix U95Av2 expression chip were assessed for expression of BAF complex members and correlations [unique npBAF members include PHF10 and ACTL6A; unique nBAF members include DPF1 (DPF3 was not on the genechip), ACTL6B and SMARCD3; neuritogenic target genes include GAP43, STMN2 and INA]. FB=fetal brain; LR/IR=low risk and intermediate risk group neuroblastoma; HR=high risk group; NA=non MYCN amplified; A=MYCN amplified; * denotes p<0.05 and ** denotes p<0.001 (Student's T-test).

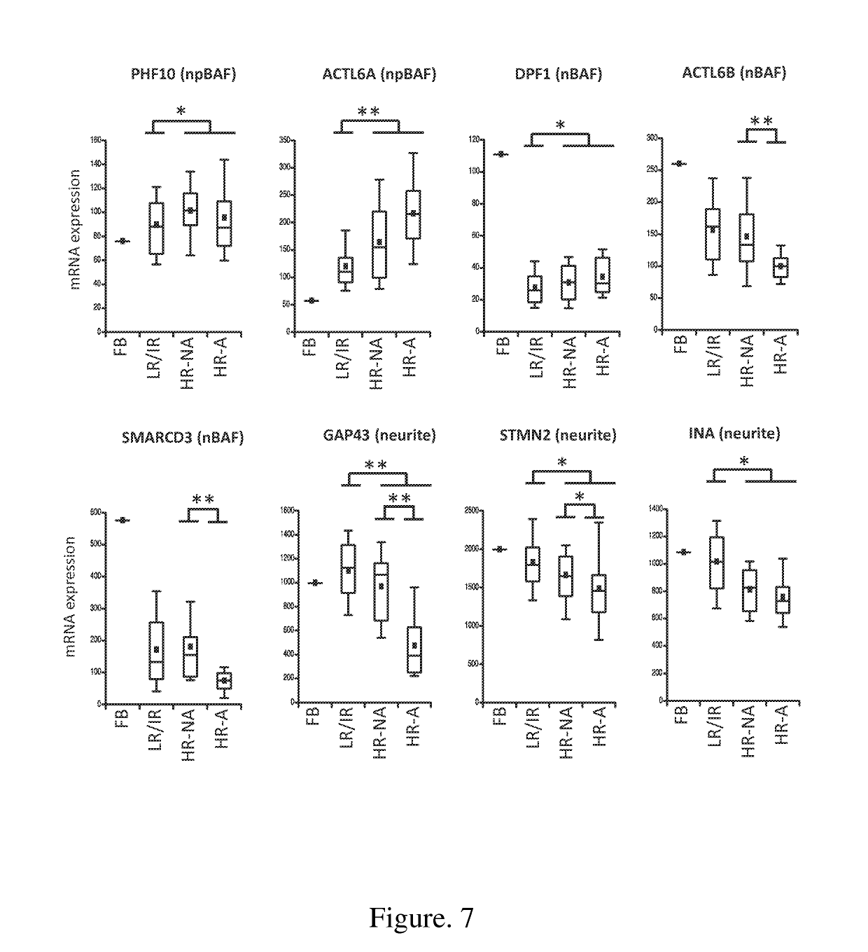

FIG. 8. (Table 1.) Summary of next generation sequencing analyses in neuroblastoma

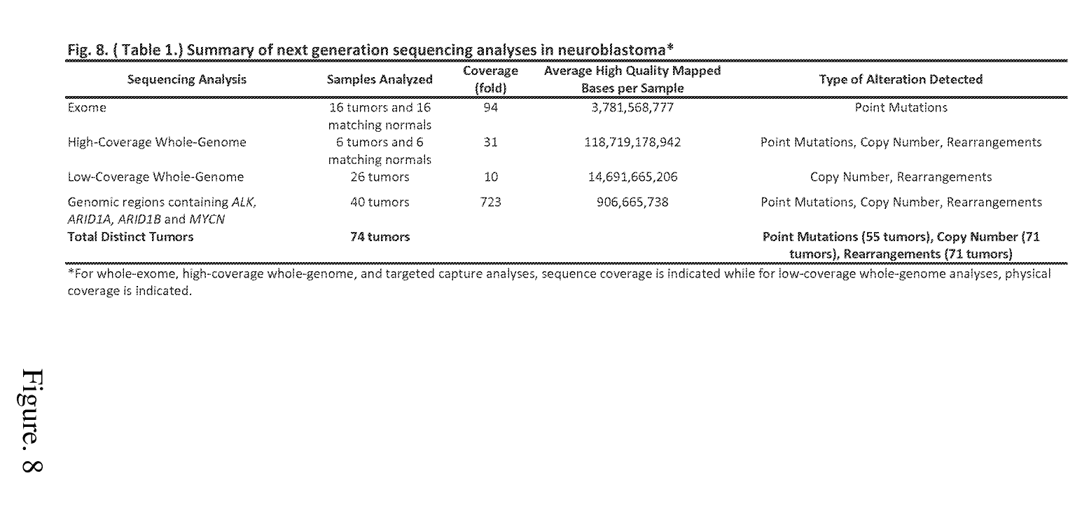

FIG. 9. (Table 2.) Summary of recurrent genomic alterations observed in neuroblastoma, including chr1:26896234 deletion (SEQ ID NO: 1).

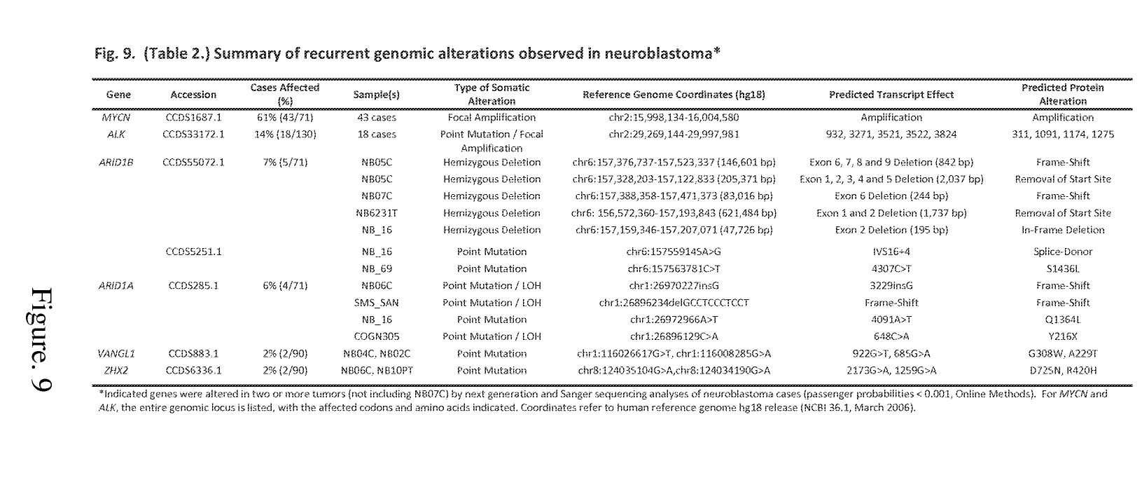

FIG. 10. (Table 3.) Biomarker Analyses in Neuroblastomas

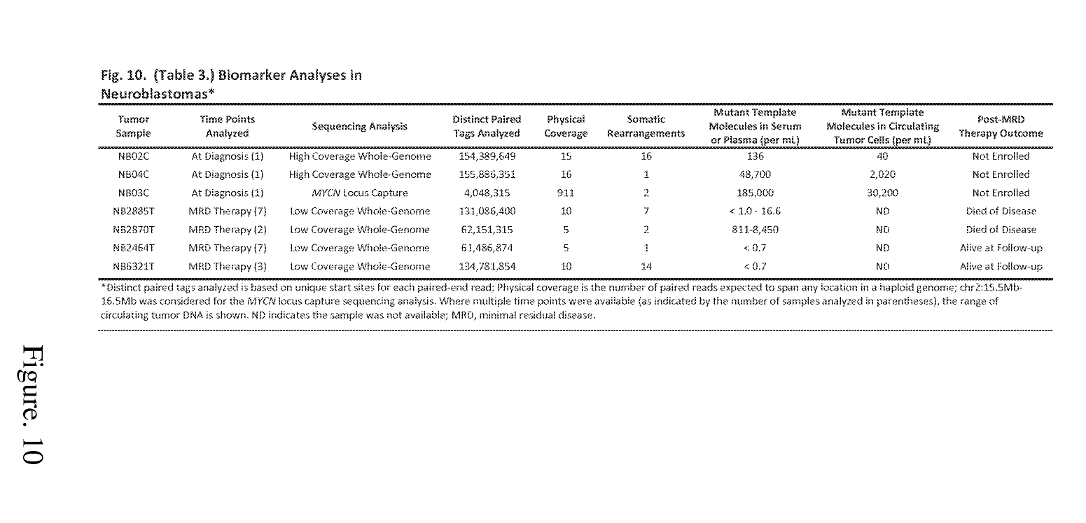

### DETAILED DESCRIPTION OF THE INVENTION

The inventors have developed methods for detecting, monitoring, and categorizing neuroblastomas. Additionally, models of the disease can be made and substances tested to assess their potential as drugs for treating neuroblastomas.

Biological samples which can be tested include without limitation blood, serum, plasma, saliva, lymph, tissue, cells, and cerebral spinal fluid.

Methods for testing for a deletion or a mutation include without limitation whole genome or targeted sequencing, exome sequencing, nucleic acid hybridization, amplification of nucleic acids, allele-specific ligation, allele specific amplification, single base extension, array hybridization, denaturing high pressure liquid chromatography (dHPLC), RFLP analysis, AFLP analysis, single-stranded conformation polymorphism analysis, an amplification refractory mutation system method, single nucleotide primer extension, oligonucleotide ligation, nucleic acid hybridization, gel electrophoresis, FRET, chemiluminescence, base excision sequence scanning, mass spectrometry, microarray analysis, linear signal amplification technology, rolling circle amplification, SERRS, fluorescence correlation spectroscopy, and single-molecule electrophoresis.

Deletions and/or mutations in ARID1B1 can be used to predict a decreased overall survival risk or presence of minimal residual disease after potentially curative therapy. The level of mutant or deleted ARID1B1 can be used as a biomarker of tumor burden or of response to therapy. Typically where levels of a biomarker such as ARID1B1 are measured, they are assessed at multiple times and compared one to another. Increases or decreases in the biomarker levels are indications of increased or decreased tumor burden and/or of lesser or greater efficacy of a treatment. To assess a treatment efficacy, one can make a measurement at a time point before and after treatment, or two points during an ongoing treatment.

Once a particular deletion or mutation has been identified in ARID1B1 in a patient, a primer or probe can be designed to specifically hybridize to the deleted or mutated nucleic acid. Such a personalized primer or probe can be used to readily assess tumor dynamics or response to therapy in the individual patient. Mutations and deletions may include missense, splice site, small deletions of 1 or 2 nucleotides, or larger deletions of 3-10, 20-50, 50-1000, or 1000-10,000 nt, for example. The mutations or deletions may map to any portion of the ARID1B1 gene, including any one or more of exons 1, 2, 3, 4, 5, 6, 7, 8, or 9. Alternatively, a pair of primers can be designed which bracket a deletion or mutation so that the mutation or deletion is present within the amplicon.

Probes may specifically hybridize or detect the following mutations or deletions in the ARID1B1 gene: a deletion in exons 6, 7, and 8; a deletion in exons 1, 2, 3, 4, and 5; a deletion in exon 6; a deletion in exons 1 and 2; a splice donor mutation at IVS16+4; a 4307C>T mutation; a frame-shift mutation, a deletion that removes the start site; an in-frame deletion; a splice-donor mutation; a mutation changing Ser1436 to Leu. Probes and primers are isolated nucleic acid molecules that are removed from their chromosomal flanks and neighbors. The removal may be accomplished by selective synthesis, for example, rather than by physical removal of flanks Typically probes and primers are purified from nucleic acids with differing sequences so that the composition is essentially homogeneous.

Mutations and deletions may also be detected by identifying abnormalities in the ARID1B1 protein. Techniques which may be used include gel electrophoresis, protein sequencing, HPLC-microscopy tandem mass spectrometry technique, immunoaffinity assay, immunoprecipitation, immunocytochemistry, ELISA, radioimmunoassay, immunoradiometry, and immunoenzymatic assay.

Detection of a mutation or deletion in ARID1B1 can be used as a classifier. It can be used to define, alone or together with other factors, arms of a clinical trial. The classifier can be used to make a therapeutic choice. The therapy may be associated with better outcome in the presence of the classifier. Alternatively, the classifier may suggest a prognosis which in turn will suggest a more aggressive or less aggressive therapy.

Treatment options for neuroblastoma include watchful waiting, surgery followed by watchful waiting, surgery followed by combination chemotherapy, radiation therapy, 13-cis retinoic acid, stem cell transplant, high-dose chemotherapy, radioactive iodine therapy, monoclonal antibody therapy, biologic therapy. The presence or absences of a mutation in ARID1B1 will guide the treatment option. Common chemotherapy drugs used to treat neuroblastoma include cyclophosphamide, cisplatin, doxorubicin, etoposide, carboplatin and vincristine. Disialoganglioside (GD2) may be used as target for immunotherapy because this antigen is expressed at a high density in the majority of human NB tumors. Several anti-GD2 monoclonal antibodies have been developed and tested in clinical trials. GM-CSF can be used inter alia to enhance anti-GD2 mediated ADCC. Interleukin-2 (IL-2) can also be used to augment lymphocyte-mediated ADCC, particularly of anti-GD2 antibodies.

Disease models can be made using somatic or germ cells in which an ARID1B1 mutation or deletion is made or inserted. The cells may be cultured in vitro. The cells may be passaged within an animal.

Candidate drugs may be without limitation any small molecule, peptide, nucleic acid, antisense molecule, antibody, antibody fragment, single chain antibody. Drugs may be selected rationally for testing or may be randomly tested. Drugs can be designed to have certain properties anticipated to be beneficial.

Similarly inhibitors of ARID1B1 can be any type of molecule which has the function of inhibiting the protein's biological function. The inhibitor may be any small molecule, peptide, nucleic acid, antisense molecule, antibody, antibody fragment, single chain antibody.

While neuroblastomas are a prevalent in childhood cancers, the same mutations may also be found and used in adult cancers, including adult neuroblastomas.

Prognoses can be provided by a written or electronic means. They can be recorded in a paper or an electronic record. They may be tentatively assigned at a clinical laboratory, prior to or in consultation with the treating physician.

Our study underscores the importance of integrated genomic analyses, including detection of sequence alterations, copy number changes, and rearrangements that can now be performed using massively parallel sequencing approaches to identify subtle genomic changes. Despite the comprehensive efforts of this study, some alterations may not have been detected. First, a small fraction of the exome was not analyzed, either due to low sequence coverage in the whole-genome analyses or inadequate capture in the exome analyses. Second, it is possible that point mutations in non-protein-coding regions of the genome may be involved in neuroblastoma. Such data were obtained for six neuroblastoma cases and did not identify any clear clustering of alterations; analysis of additional neuroblastoma cases could be useful to further interpret these non-coding changes. Third, germline neuroblastoma susceptibility variants have been identified^{43,44} and additional such variants yet to be discovered may be present in our neuroblastoma cases. Fourth, it is possible that epigenetic alterations contribute to the initiation or progression of neuroblastomas. This possibility is intriguing given the new data on ARID1B and ARID1A in this tumor type. Finally, although rearrangements and copy number changes were detected in a genome-wide fashion, many of these occurred in non-coding regions and their functional roles remain to be elucidated.

Our data add to the growing knowledge of the genomic landscapes of human cancers. They are consistent with the idea that pediatric tumors do not require as many genetic alterations as typical adult cancers^{13,45}. Although few alterations were identified in known therapeutically-targetable oncogenes such as ALK, there are many other alterations, both subtle and large, that are found in these cancers and many of these affect chromatin-modifying genes. These data highlight the important connection between genetic alterations in the cancer genome and epigenetic pathways, and provide new avenues for research and disease management in neuroblastoma patients.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Whole-Exome and Whole-Genome Next Generation Sequencing Analyses

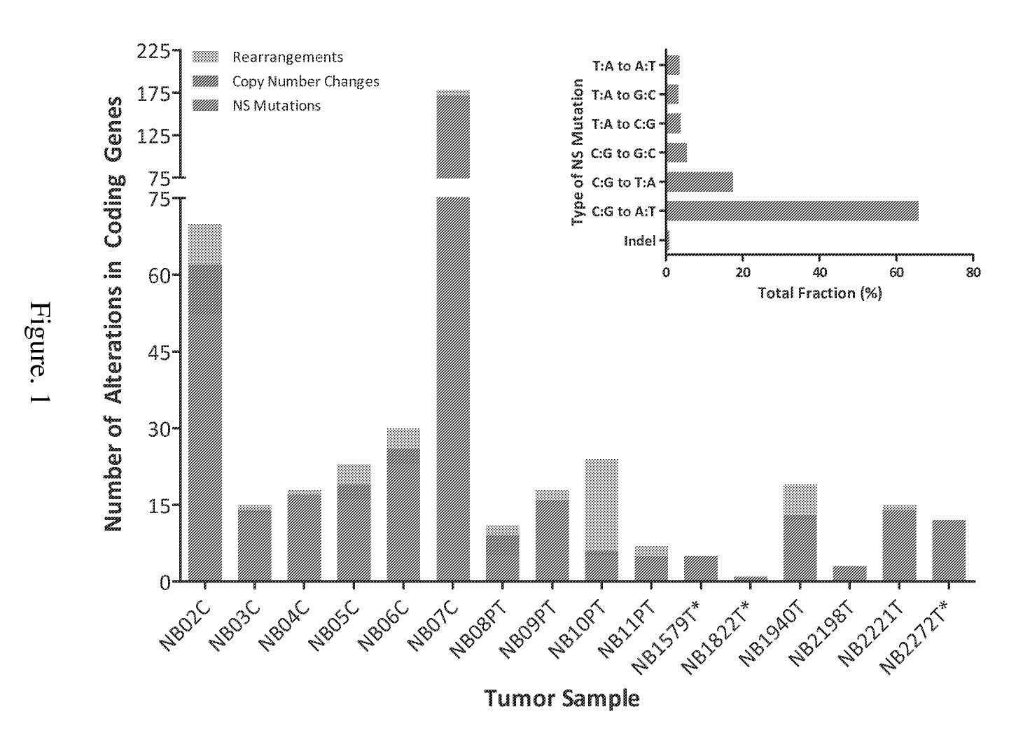

To comprehensively analyze acquired genetic alterations in neuroblastoma, we used a combination of next generation sequencing approaches in a discovery screen: low-coverage whole-genome sequencing for detection of structural and copy number alterations in 26 cases; exome sequencing for detection of subtle sequence alterations in 16 cases; and high-coverage whole-genome sequencing for detection of both sequence and structural alterations in 6 cases (all of which were also subjected to exome sequencing) (Supplementary FIG. 1, Table 1). In total, 16 cases could be analyzed for subtle mutations such as single base substitutions and small insertions or deletions (indels), while 32 cases (26 with low coverage, 6 with high coverage) could be analyzed for large scale structural changes and copy number alterations. DNA was obtained from low-passage cell lines (n=6) or primary tumors (n=29) and matched normal controls as indicated in Supplementary Table 1. Following library construction and capture on a SureSelect (Agilent) Enrichment System, DNA was sequenced using Illumina GAIIx/HiSeq instruments (Supplementary Note). The average coverage of each base in the targeted regions was 31-fold and 94-fold for the high-coverage whole-genome and exome sequencing approaches, respectively (Supplementary Tables 2 and 3), while the low-coverage whole genomic sequencing achieved an average of 10-fold physical coverage (Supplementary Table 4).

The sequencing data were analyzed using stringent criteria to identify somatic single base substitutions, insertions or deletions (indels), and structural alterations (Online Methods). All single base substitutions and indels were confirmed by an independent sequencing method (Online Methods), and only confirmed mutations are included in the analyses described below. With the exception of one tumor, we found that neuroblastoma tumors had an average of 13 (range, 1 to 52) somatically acquired single base substitution or indel mutations that would be predicted to result in non-silent (NS) changes in coding regions. The NS substitutions were predominantly C:G to A:T transversions (FIG. 1; Supplementary Table 5), representing a mutation spectra different from other pediatric and adult tumors^{13,14,15}. Overall, we detected 368 mutations in 353 genes (Supplementary Table 5). The average number of somatic mutations in neuroblastomas was similar to that reported for neuroblastoma by Molenaar¹⁶ and slightly higher than the number in medulloblastomas, a pediatric tumor analyzed by exome sequencing¹³. This is notably lower than the number of alterations observed in most common adult solid tumors^{14,15}. One tumor-derived cell line, NB07C, had a substantially higher number of somatic mutations (169 NS changes) than the other neuroblastomas analyzed. This case was considered to be an outlier in this study but may identify a unique subset of cases if similar tumors are identified in future validation efforts.

Six samples were analyzed by both exome and high-coverage whole-genome sequencing, permitting independent validation of the somatic alterations as well as a comparison of these approaches for the detection of sequence alterations. Over 91% of the whole-genome and 94% of whole-exome targeted bases were represented by at least 10 reads (Supplementary Tables 2 and 3). A total of 245 somatic alterations in coding regions were detected by either approach with 219 mutations identified by whole-genome sequencing and 240 alterations identified by whole-exome sequencing. Exomic and genomic sequencing detected 98% and 89%, respectively, of the mutations, consistent with similar comparisons made by others¹⁷.

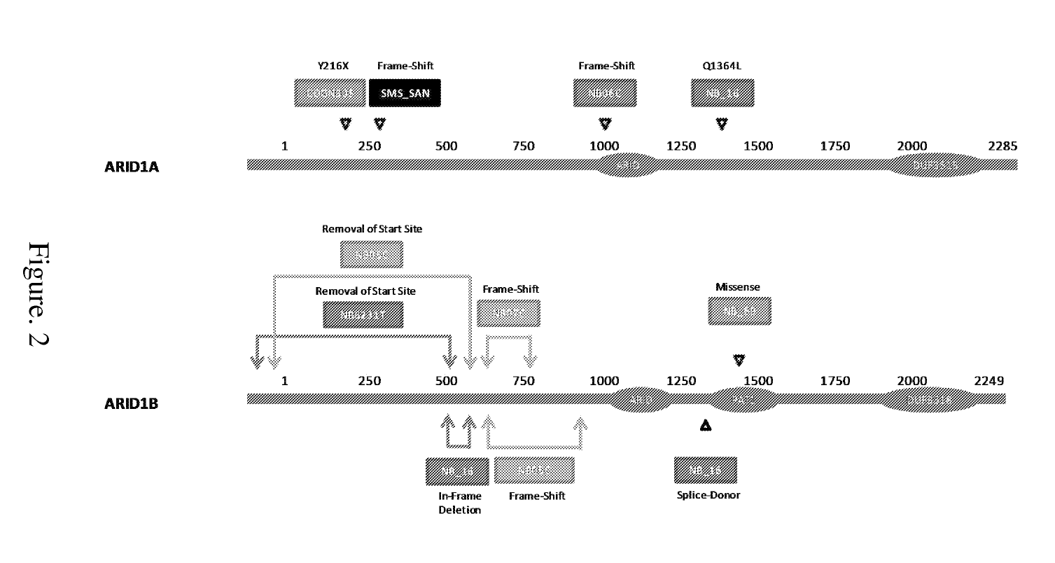

In addition to the single base substitutions and indels, we analyzed copy number changes corresponding to focal amplifications (≧5-fold copy number gain) or homozygous deletions (less than 20 Mb in size) as these are likely to harbor potential oncogenes and tumor suppressor genes. There was an average of two such focal copy number changes per tumor (range, 0 to 10 per tumor) whose boundaries included at least one protein-encoding gene (Supplementary Table 6); all were amplification events and the majority included either MYCN or ALK as the putative target gene. One tumor amplicon (in NB1395T) harbored LIN28B, which is downstream of MYCN and a putative neuroblastoma oncogenic driver^{18,19}. There were also four structural rearrangements per tumor that were within protein-encoding genes (range, 0 to 18 per tumor; Supplementary Tables 4 and 7 and Supplementary FIG. 2). These included deletions, duplications, and inversions within the same chromosome as well as inter-chromosomal translocations. We did not find evidence of chromothrypsis in these samples, although this has recently been reported in a subset of high-risk neuroblastoma tumors¹⁶.

### EXAMPLE 2

### Candidate Neuroblastoma Driver Genes and Targeted Sequencing Analyses

The coding exons of all genes that were recurrently altered in the tumors analyzed by next generation sequencing were examined by PCR and Sanger sequencing in 74 additional neuroblastoma cases (Table 2, Supplementary Table 1 and Online Methods). Integration of these data with next generation sequencing data revealed a number of novel genes as well as those previously known to be involved in neuroblastoma. The ALK receptor tyrosine kinase gene was found to be mutated in 8 of 90 cases (9%) in our discovery screen (Table 2 and Supplementary Table 5). All eight sequence changes in ALK affected two amino acid residues in the tyrosine kinase domain (R1275Q, R1275L and F1174L) that have been reported to lead to constitutive kinase activity^{4,8,11}. An additional 15-fold amplification of the ALK gene was identified in one of 32 cases evaluated for structural changes and copy number alterations (Supplementary Table 6). However, no ALK translocations were detected, suggesting that this mechanism of ALK activation, typical of large cell lymphomas, non-small cell lung cancers, and inflammatory myofibroblastic tumors, is uncommon in neuroblastoma^{20,21}. Additionally, the MYCN oncogene was found to be focally amplified in 15 of the 32 (47%) neuroblastomas, including 5 of the 6 neuroblastoma cell lines, consistent with the previously reported frequency of MYCN amplification in high risk tumors and cell lines derived from such tumors⁷ (Table 2 and Supplementary Table 6). Co-amplification of ODC1, a MYCN target gene important for oncogenicity in neuroblastoma²², was seen in 3 of 15 (20%) MYCN amplified tumors (none of which displayed copy number changes of ALK). Other alterations in known cancer genes included a glutamine to lysine change at codon 61 in the HRAS oncogene, and single missense alterations in the PTCH1 tumor suppressor and in the EGF receptor family member ERBB4 (Supplementary Table 5).

In addition to these alterations, a number of mutations in genes not previously known to be involved in neuroblastoma were identified. The most prominent example was the detection of intragenic hemizygous deletions targeting the AT rich interactive domain 1B gene, ARID1B, in three of 32 tumors (9%) in the discovery screen (FIG. 2, Table 2, and Supplementary Table 7). The deletions in ARID1B were identified by virtue of their aberrantly spaced paired-end sequences and, due to their small size and hemizygous nature, would have been difficult to detect using conventional copy number analyses. These included an 83 kb deletion encompassing exon 6 and a 147 kb deletion encompassing exons 6-9 that were predicted to result in a frameshift and premature truncation of the gene products, and a 621 kb deletion that removed exons 1 and 2, including the protein translation start site (FIG. 2 and Table 2). All these deletions, which were confirmed by PCR amplification and sequencing across the deletion junction, would be expected to abolish functional translation of the key downstream DNA binding (ARID) and topoisomerase-II associated (PAT1) protein domains of ARID1B. An additional tumor had an insertion mutation in the homologous ARID1A gene that would be predicted to lead to premature termination of the protein.

To investigate the prevalence of these specific alterations identified in the discovery screen, we designed a custom capture approach to selectively sequence and detect point mutations and structural alterations in the genomic regions of ARID1A, ARID1B, ALK and MYCN in 40 additional neuroblastoma cases (Supplementary FIG. 1, Prevalence Screen). These analyses yielded an average sequence coverage of 723-fold per targeted base (Supplementary Tables 1 and 8). Through these analyses we were able to identify an intragenic hemizgyous deletion, a splice-site mutation and a missense mutation in ARID1B in two additional tumors as well as an additional intragenic deletion in a previously analyzed sample (NB05) (FIG. 2, Table 2 and Supplementary Tables 5 and 7). Collectively, ARID1B point mutations or intragenic deletions were identified in 5/71 (7%) of neuroblastoma cases (FIG. 2 and Table 2). We further identified hemizygous deletions encompassing the entire coding region of ARID1B in the distal region of 6q in 5 additional cases (Supplementary Table 6). Furthermore, point mutations of ARID1A were identified in three additional cases, two of which led to biallelic inactivation through mutation predicted to result in premature termination of the protein and deletion of the alternative allele at 1p36 (FIG. 2 and Table 2, Supplementary Table 5). All of these alterations were confirmed by Sanger sequencing. Not surprisingly, we identified additional ALK missense changes and MYCN amplifications, resulting in somatic alterations of ALK in 18/130 (14%) and of MYCN in 43/71 (61%) of total cases (Table 2, Supplementary Tables 5 and 6).

ARID1B is a member of the SWI/SNF transcriptional complex that is thought to regulate chromatin structure²³. Mutations recently identified in ARID1B suggest that it may serve as a potential tumorigenic driver in a small fraction of hepatocellular²⁴, breast²⁵, ovarian²⁶, and medulloblastoma^{27,28} tumors. Through our integrated genomic analyses, our findings of five independent structural alterations and two sequence changes, the majority of which would result in a truncated protein, strongly support this gene as a contributor to neuroblastoma oncogenesis (passenger probability P<0.001). Interestingly, we found sequence alterations in other genes involved in chromatin regulation in neuroblastoma. These included two frameshift, one nonsense and one missense mutation in ARID1A, another SWI/SNF complex member, nonsense mutations in the histone acetyl transferase (HAC) genes EP300 and CREBBP, and missense mutations in the SWI2/SNF2 family member TTF2 gene, the histone demethylase gene KDM5A, and the chromatin remodeling zinc finger gene IKZF1. Genes involved in chromatin structure or remodeling have been reported to be implicated in human cancers. These include a high frequency of alterations of ARID1A in ovarian clear cell carcinomas²⁶, SMARCB1 in malignant rhabdoid tumors²⁹, alterations of PBRM1 in renal cell carcinomas³⁰, alterations of EP300 and CREBBP in transitional cell carcinomas of the bladder³¹ and B cell lymphomas³², alterations of DAXX and ATRX in pancreatic endocrine tumors³³, and inactivation of histone methyltransferases MLL2 and MLL3 in medulloblastomas¹³, among others³⁴⁻³⁶. Of note, ATRX has recently been shown to be mutated in neuroblastoma tumors from adolescents and young adults (≧12 years old)¹² but would not have been expected to be altered in a significant fraction of the patients evaluated in our study (median age of diagnosis <2 years old, range <1 to 6 years old).

### EXAMPLE 3

### Personalized Genomic Biomarkers for Neuroblastoma Patients

Although the number of sequence alterations in neuroblastomas was low compared to adult tumors, the frequency of recurrent structural rearrangements in neuroblastomas was relatively high. Every tumor had at least one rearrangement (range, 1 to 66) and all cases that had recurrent copy number changes of the MYCN, ARID1B, or ALK genes also had rearrangements at these loci. Such rearrangements are not present in normal cells and could therefore be useful as biomarkers of neuroblastoma. Given the poor treatment outcomes of many neuroblastoma patients, the availability of non-invasive biomarkers to detect minimal residual disease after surgery and to measure molecular response to chemotherapy would be useful for clinical management of neuroblastoma patients.

To demonstrate the feasibility of this approach, we developed personalized biomarkers based on the rearrangements present in the cancers analyzed³⁷. This was performed through analysis of either whole-genome sequencing or capture and sequencing of the MYCN locus to identify structural alterations associated with novel rearrangement junctions not present in the germline (Online Methods). We have previously shown that tumor-specific rearrangements have the potential to serve as highly sensitive biomarkers for tumor detection and monitoring³⁷, and would therefore be expected to have fundamental advantages over measurement of wild-type sequences, including wild-type MYCN levels³⁸, in neuroblastoma patients. Notably, both MYCN amplified and non-amplified tumors had identifiable somatic rearrangement biomarkers, and in three cases in which serum was available at the time of diagnosis, we were able to detect and quantify such specific tumor rearrangements in the patients' serum (Table 3, Supplementary Table 9). Interestingly, quantitative analyses showed that there was much more tumor DNA freely floating in the serum than in circulating cells, suggesting that the cell free compartment of blood may represent a more sensitive source for detection of tumor burden (Table 3).

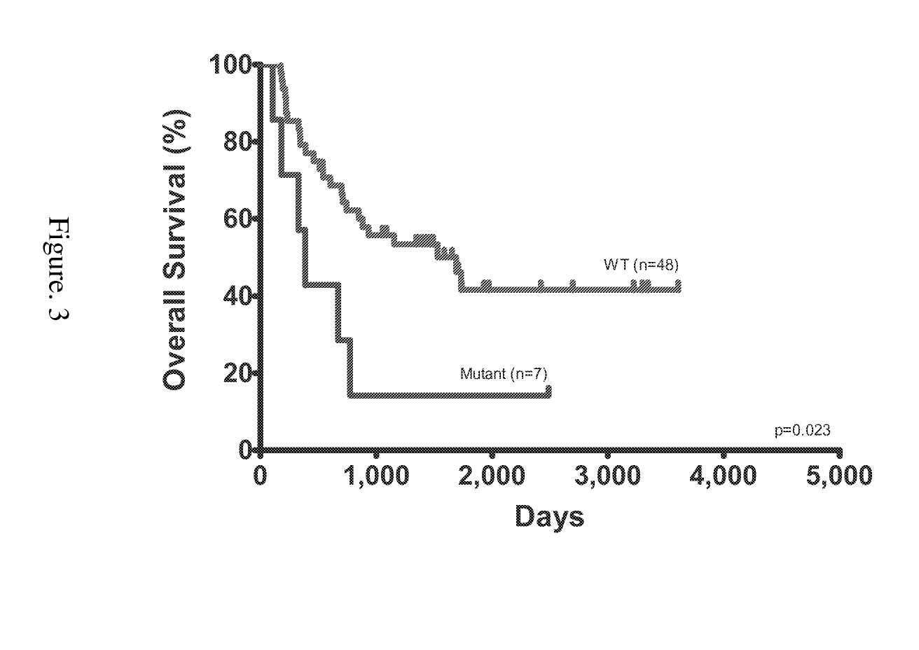

We developed personalized rearrangement biomarkers to monitor circulating tumor DNA (ctDNA) in serial plasma samples from four additional cases of neuroblastoma obtained during a post-consolidation minimal residual disease (MRD) immunotherapy trial³⁹ (Supplementary FIG. 3). In two cases, NB2885T and NB2870T, the ctDNA was detected at the end of standard high risk neuroblastoma therapy and, despite MRD immunotherapy, went on to relapse and eventually die of disease. The prolonged reduction in ctDNA in NB2885T during immunotherapy may be an indication of therapeutic response whereas the marked increase in ctDNA in NB2870T correlated with clinical relapse during the trial period. In cases NB6321T and NB2464T, no ctDNA was detectable and these patients were alive at the last follow-up over one and four years later, respectively. These data demonstrate that ctDNA may be a useful surrogate for the level of clinical disease, and that the presence of ctDNA may be a highly sensitive and specific predictor of minimal residual disease and subsequent relapse⁴⁰.

### EXAMPLE 4

### ARID1 Alterations and Clinical Correlates

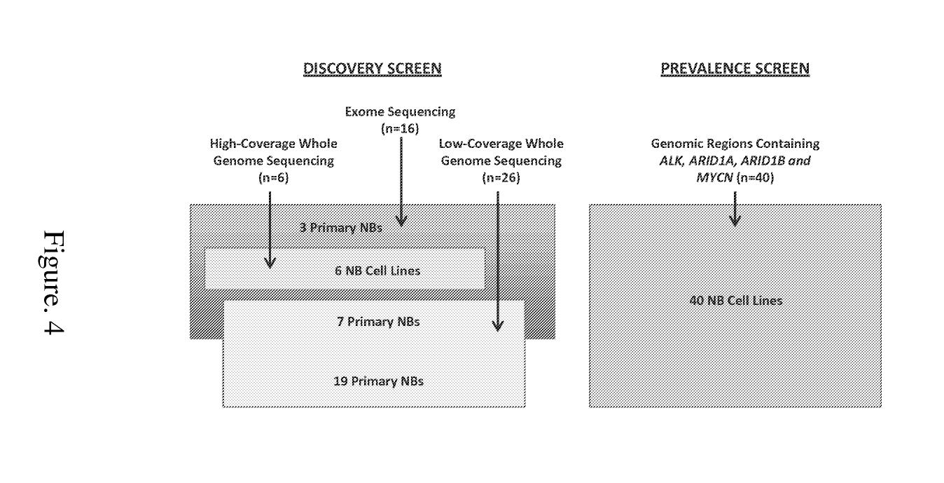

These genome-wide sequence analyses suggest that neuroblastoma tumors are driven by a relatively small number of somatically acquired alterations and that genes involved in chromatin remodeling, including ARID1B and ARID1A, were enriched for alterations. ARID1 family genes are integral components of the SWI/SNF neural progenitors-specific chromatin remodeling BAF complex that is essential for the self-renewal of multipotent neural stem cells⁴¹. Tumor-specific deletions encompassing ARID1B have been reported in CNS tumors⁴² and multiple members of this complex have been identified as tumor suppressor genes^{26,41}. We found that high expression of members unique to the neural-progenitor BAF complex correlates with a high-risk neuroblastoma phenotype while high expression of those specific to the neuron specific BAF complex, or downstream neuritogenesis target genes, correlates with lower risk neuroblastoma (Supplementary FIG. 4). These data support a model whereby disrupted BAF complex signaling may preserve an undifferentiated progenitor state.

The model above would suggest that alterations in ARID1 may correlate with a more aggressive neuroblastoma phenotype. All but one of the patients with alterations in ARID1A or ARID1B died of progressive disease, including a child with low-risk neuroblastoma (a group with a survival probability of >98%). ARID1 alterations were associated with inferior overall survival of 386 days compared to 1689 days for patients without such alterations (hazard ratio, HR 4.49; 95% confidence interval, CI 1.24-16.33; P=0.0226, log-rank test; FIG. 3 and Supplementary Table 10). An analysis that also included hemizygous deletions of the entire coding region of ARIDB further increased the significance of the survival difference between patients with mutant and wildtype ARID1B/A (hazard ratio, HR 6.41; 95% confidence interval, CI 1.93-21.25; P=0.0024, log-rank test). The median survival of patients with ARID1 alterations was lower than that of any other genetic alterations assessed, including MYCN amplification (median survival 726 days) providing a potential marker for early therapy failure and disease progression.

### EXAMPLE 5

### Samples Obtained for Sequencing Analyses

Neuroblastoma tumor DNA (from cell lines and primary tumors), matched germline DNA (from peripheral blood or lymphoblastoid cell line) and patient serum or plasma were obtained from the Children's Oncology Group (COG) cell line repository and the COG Neuroblastoma biobank following committee approval (study #COG NB 2008-02). Informed consent for research use was obtained from all patients and/or parents at the enrolling COG member institution prior to tissue banking or cell line generation, and study approval was obtained from The Children's Hospital of Philadelphia Institutional Review Board. All samples were STR genotyped to confirm identity. Primary tumor samples were selected from patients with COG high-risk disease, and specimens verified to have >75% viable tumor cell content by histopathology assessment. Serial plasma samples for MRD assays were obtained from patients enrolled on the COG ANBL0032 immunotherapy study.

### EXAMPLE 6

### Massively Parallel Paired-End Sequencing and Somatic Mutation Identification

Genomic DNA libraries were prepared and captured following Illumina's (Illumina, San Diego, Calif.) suggested protocol with the modifications described in the Supplementary Note, or by Personal Genome Diagnostics (Baltimore, Md.). DNA libraries were sequenced with the Illumina GAIIx/HiSeq Genome Analyzer, yielding 100 or 200 base pairs of sequence from the final library fragments for high coverage exome/low coverage genome and high coverage genome analyses respectively. Sequencing reads were analyzed and aligned to human genome hg18 with the Eland algorithm in CASAVA 1.7 software (Illumina). Reads were mapped using the default seed-and-extend algorithm, which allowed a maximum of 2 mismatched bases in the first 32 bp of sequence. Identification of somatic alterations was performed as previously described⁴⁶⁻⁴⁹ utilizing a next-generation sequencing analysis pipeline that enriched for tumor-specific single nucleotide alterations and small insertions/deletions. Briefly, for each position with a mismatch (as compared to the hg18 reference sequence using the Eland algorithm) the read coverage of the mismatch and wild-type sequence at that base was calculated. A candidate mismatched base was identified as a mutation only when (i) two or more distinct paired-tags contained the mismatched base; (ii) the number of distinct paired-tags containing a particular mismatched base was at least 7.5% of the total distinct tags; and (iii) the mismatched base was not present in >0.5% of the tags in the matched normal sample. Candidate somatic point mutations identified by next generation sequencing approaches were confirmed by an independent sequencing method (either a different next-generation sequencing approach or polymerase chain reaction (PCR) followed by Sanger sequencing, Supplementary Table 5).

### EXAMPLE 7

### Evaluation of Genes in Additional Tumors and Matched Normal Controls

For 12 selected genes that were somatically altered, the coding region was sequenced in a validation set composed of an independent series of 74 additional neuroblastomas and matched controls. These genes included ALK, ANKRD34B, ARID1B, ARID1A, FAR1, PRSS16, PRSS23, RASGRP3, TTLL6, VANGL1, VCAN and ZHX2. PCR amplification and Sanger sequencing analyses were performed following protocols described previously¹⁵.

### EXAMPLE 8

### Identification of Somatic Copy Number Alterations

Single tags passing filter were grouped by genomic position in nonoverlapping 3-kb bins. A tag density ratio was calculated for each bin by dividing the number of tags observed in the bin by the average number of tags expected to be in each bin (on the basis of the total number of tags obtained for chromosomes 1 to 22 for each library divided by 849,434 total bins). The tag density ratio thereby allowed a normalized comparison between libraries containing different numbers of total tags. A control group of libraries made from the six matched normal high coverage whole-genome samples from Supplementary Table 1 and six additional normal samples [Co84N, Co108N, B5N, B7N³⁷ and CEPH (Centre d'Etude du Polymorphisme Humain) samples NA07357 and NA18507] was used to define areas of germline copy number variation or that contained a large fraction of repeated or low-complexity sequences. Any bin where at least two of the normal libraries had a tag density ratio of <0.25 or >1.75 was removed from further analysis.

For all samples analyzed with low coverage whole-genome sequencing (Supplementary Table 4), amplifications were identified as three or more bins with tag ratios of >2, separated by no more than ten intervening bins with a tag ratio <2. For all amplifications, at least one bin had a tag ratio of ≧5. For samples with high coverage whole-genome sequencing (Supplementary Table 3), homozygous deletions were identified as three or more bins with tag ratios of <0.25, separated by no more than ten intervening bins with a tag ratio >0.25. Single-copy gains and losses were identified through visual inspection of tag density data for each sample.

For all samples analyzed with targeted capture sequencing, the tag ratio for each gene was calculated as the average read coverage for the gene, divided by the average read coverage of the ALK, ARID1A and ARID1B genes (MYCN was not used as it is frequently amplified). These values were normalized to the average coverage for each gene in a normal sample. Amplifications and hemizygous deletions were identified if the tag ratio for a gene was ≧5.0 or <0.65, respectively. Hemizgyous deletions were confirmed through LOH analyses of SNPs in the genomic region of each gene.

Six samples with high coverage whole-genome sequencing were analyzed for amplifications at the MYCN locus. The boundary coordinates for these amplifications were compared and a one megabase (hg18 chr2:15.5 Mb-16.5 Mb) region was identified that contained at least one amplification boundary region from each sample.

### EXAMPLE 9

### Identification of Somatic Rearrangements

Somatic rearrangements were identified by querying aberrantly mapping reads from one flow cell of an Illumina GAIIx run (100 bp PE) or up to two lanes of an Illumina HiSeq Genome Analyzer run (50 bp PE) to achieve a physical coverage of >8X. The discordantly mapping pairs were grouped into 1 kb bins when at least 2 distinct tag pairs (with distinct start sites) spanned the same two 1 kb bins (known bins which contained aberrantly mapping tags were removed as described above³⁷, as well as 1 kb bins involved in known germline structural alterations⁵⁰).

To identify all high-confidence genomic rearrangements, candidate rearrangements were filtered using the above described criteria and were required to have at least one tag sequenced across the rearrangement breakpoint. Breakpoints were determined using

BLAT alignment to the human genome sequence (hg18)⁵¹. In order to ensure that no recurrent rearrangements in coding genes were missed, genes which harbored rearrangements were evaluated for all candidate rearrangements without the requirement that the breakpoint be present in a sequenced tag and any recurrent gene rearrangement was further analyzed. Candidate rearrangements were confirmed as somatic when a 10 uL PCR based reaction (containing 5.9 uL H₂O, 1 uL 10×PCR buffer, 1 uL 10 mM dNTPs, 0.6 uL DMSO, 0.4 uL 25 uM primers, 0.1 uL Platinum Taq and 1 uL DNA, 3 ng/uL) resulted in the amplification of a product of the expected size in the tumor but not in the matched normal on a 1% ethidium bromide stained agarose gel. Utilizing this stringent pipeline, of the 26 candidate genomic rearrangements tested, 25 were confirmed as somatic (96%) as well as 15 of the 16 candidate rearrangements tested that were identified by the NMYC capture sequencing method (94%). In all three cases of ARID1B somatic rearrangement, the PCR product was Sanger sequenced to identify the breakpoint to the base-pair resolution. For biomarker analyses, rearrangements were identified with the initial-above described method, with a subsequent PCR product sequenced and aligned using BLAT to hg18⁵¹ in order to design primers to amplify a PCR product in the serum, plasma or peripheral blood between 70 and 120 bp.

### EXAMPLE 10

### Quantification of Tumor Burden in Serum and Peripheral Blood

Circulating tumor DNA was amplified using 2× Phusion Flash PCR Master Mix and patient specific primers (at a final concentration of 0.5 uM each) in DNA isolated from serum or plasma and DNA isolated from peripheral blood cells. Subsequently, the level of tumor DNA was quantified after amplification by digital PCR on SYBR green I stained 10% TBE gels³⁷.

### EXAMPLE 11

### Gene Expression Analyses

For gene expression profiling by Affymetrix U95Av2 microarrays, the expression measures for each probe set was extracted and normalized using robust multi-array average protocols from raw CEL files as described previously. Basic linear correlation and regression was used to define r, r² and two-tailed p value to assess correlation among gene expression values.

### EXAMPLE 12

### Statistical Analyses for Clinical and Genetic Data

Curves for overall survival (calculated as the time from diagnosis) were constructed using the Kaplan-Meier method and compared between groups using the log-rank test for descriptive purposes. Cox proportional hazards models were used to test for the effect of clinical and genetic parameters on survival. Passenger probabilities were calculated using the binomial test adjusted for gene sizes and corrected for multiple comparisons⁵².

### EXAMPLE 13

### Preparation of Next-Generation Sequencing Libraries

Illumina genomic DNA libraries were prepared for massively parallel paired-end sequencing with the following steps: (1) 1-3 micrograms (μg) of genomic DNA from tumor or peripheral blood in 100 microliters (μl) of TE was fragmented in a Covaris sonicator (Covaris, Woburn, Mass.) to a size of 150-450 bp. To remove fragments smaller than 150 bp, DNA was mixed with 25 μl of 5× Phusion HF buffer, 416 μl of ddH₂O, and 84 μl of NT binding buffer and loaded into NucleoSpin column (cat# 636972, Clontech, Mountain View, Calif.). The column was centrifuged at 14,000 g in a desktop centrifuge for 1 min, washed once with 600 μl of wash buffer (NT3 from Clontech), and centrifuged for 1 min and again for 2 min to dry completely. DNA was eluted in 45 μl of elution buffer included in the kit. (2) Purified, fragmented DNA was mixed with 40 μl of H₂O, 10 μl of End Repair Reaction Buffer, 5 μl of End Repair Enzyme Mix (cat# E6050, NEB, Ipswich, Mass.). The 100 μl end-repair mixture was incubated at 20° C. for 30 min, purified with a PCR purification kit (Cat # 28104, Qiagen) and eluted with 45 μl of elution buffer (EB). (3) To A-tail, 42 μl of end-repaired DNA was mixed with 5 μl of 10×dA Tailing Reaction Buffer and 3 μl of Klenow (exo-) (cat# E6053, NEB, Ipswich, Mass.). The 50 μl mixture was incubated at 37° C. for 30 min before DNA was purified with a MinElute PCR purification kit (Cat # 28004, Qiagen). Purified DNA was eluted with 25 μl of 70° C. EB. (4) For adaptor ligation, 25 μl of A-tailed DNA was mixed with 10 μl of PE-adaptor (Illumina), 10 μl of 5× Ligation buffer and 5 μl of Quick T4 DNA ligase (cat# E6056, NEB, Ipswich, Mass.). The ligation mixture was incubated at 20° C. for 15 min. (5) To purify adaptor-ligated DNA, 50 μl of ligation mixture from step (4) was mixed with 200 μl of NT buffer and cleaned up with a NucleoSpin column. DNA was eluted in 50 μl elution buffer. (6) To obtain an amplified library, nine PCRs of 50 μl each were set up, each including 29 μl of H₂O, 10 μl of 5× Phusion HF buffer, 1 μl of a dNTP mix containing 10 mM of each dNTP, 2.5 μl of DMSO, 1 μl of Illumina PE primer #1, 1 μl of Illumina PE primer #2, 0.5 μl of Hotstart Phusion polymerase, and 5 μl of the DNA from step (5). The PCR program used was: 98° C. for 2 minutes; 6 cycles of 98° C. for 15 seconds, 65° C. for 30 seconds, 72° C. for 30 seconds; and 72° C. for 5 min. To purify the PCR product, 450 μl PCR mixture (from the nine PCR reactions) was mixed with 900 μl NT buffer from a NucleoSpin Extract II kit and purified as described in step (1). Library DNA was eluted with 70° C. elution buffer and the DNA concentration was estimated by absorption at 260 nm. Libraries undergoing capture of the MYCN region (hg18, chr2:15.5 Mb-16.5 Mb) were subsequently captured with probes specific to this locus.

Capture of human exome was performed following a protocol from Agilent's SureSelect Paired-End Target Enrichment System (Agilent, Santa Clara, Calif.) with the following modifications or for targeted regions by Personal Genome Diagnostics (Baltimore, Md.). (1) A hybridization mixture was prepared containing 25 μl of SureSelect Hyb # 1, 1 μl of SureSelect Hyb # 2, 10 μl of SureSelect Hyb # 3, and 13 _{I}A of SureSelect Hyb # 4. (2) 3.4 μl (0.5 μg) of the PE-library DNA described above, 2.5 μl of SureSelect Block #1, 2.5 μl of SureSelect Block #2 and 0.6 μl of Block #3; was loaded into one well in a 384-well Diamond PCR plate (cat# AB-1111, Thermo-Scientific, Lafayette, Colo.), sealed with microAmp clear adhesive film (cat# 4306311; ABI, Carlsbad, Calif.) and placed in a GeneAmp PCR system 9700 thermocycler (Life Sciences Inc., Carlsbad Calif.) for 5 minutes at 95° C., then held at 65° C. (with the heated lid on). (3) 25-30 μl of hybridization buffer from step (1) was heated for at least 5 minutes at 65° C. in another sealed plate with the heated lid on. (4) 5 μl of SureSelect Oligo Capture Library, 1 μl of nuclease-free water, and 1 μl of diluted RNase Block (prepared by diluting RNase Block 1:1 with nuclease-free water) were mixed and heated at 65° C. for 2 minutes in another sealed 384-well plate. (5) While keeping all reactions at 65° C., 13 μl of Hybridization Buffer from Step (3) was added to the 7 μl of the SureSelect Capture Library Mix from Step (4) and then the entire contents (9 μl) of the library from Step (2). The mixture was slowly pipetted up and down 10 times. (6) The 384-well plate was sealed tightly and the hybridization mixture was incubated for 22-24 hours at 65° C. with a heated lid.

After hybridization, five steps were performed to recover and amplify the captured DNA library: (1) Magnetic beads for recovering captured DNA: 50 μl of Dynal MyOne Streptavidin C1 magnetic beads (Cat # 650.02, Invitrogen Dynal, AS Oslo, Norway) was placed in a 1.5 ml microfuge tube and vigorously resuspended on a vortex mixer. Beads were washed three times by adding 200 μl of SureSelect Binding buffer, mixed on a vortex for five seconds, then removing and discarding supernatant after placing the tubes in a Dynal magnetic separator. After the third wash, beads were resuspended in 200 μl of SureSelect Binding buffer. (2) To bind captured DNA, the entire hybridization mixture described above (29 μl ) was transferred directly from the thermocycler to the bead solution and mixed gently; the hybridization mix /bead solution was incubated an Eppendorf thermomixer at 850 rpm for 30 minutes at room temperature. (3) To wash the beads, the supernatant was removed from beads after applying a Dynal magnetic separator and the beads were resuspended in 500 μl SureSelect Wash Buffer #1 by mixing on a vortex mixer for 5 seconds and incubated for 15 minutes at room temperature. Wash Buffer#1 was then removed from the beads after magnetic separation. The beads were further washed three times, each with 500 μl pre-warmed SureSelect Wash Buffer #2 after incubation at 65° C. for 10 minutes. After the final wash, SureSelect Wash Buffer #2 was completely removed. (4) To elute captured DNA, the beads were suspended in 50 μl SureSelect Elution Buffer, vortex-mixed and incubated for 10 minutes at room temperature. The supernatant was removed after magnetic separation, collected in a new 1.5 ml microcentrifuge tube, and mixed with 50 μl of SureSelect Neutralization Buffer. DNA was purified with a Qiagen MinElute column and eluted in 17 μl of 70° C. EB to obtain 15 μl of captured DNA library. (5) The captured DNA library was amplified in the following way: Seven 30 uL PCR reactions each containing 19 μl of H₂O, 6 μl of 5× Phusion HF buffer, 0.6 μl of 10 mM dNTP, 1.5 μl of DMSO, 0.30 μl of Illumina PE primer #1, 0.30 μl of Illumina PE primer #2, 0.30 μl of Hotstart Phusion polymerase, and 2 μl of captured exome library were set up. The PCR program used was: 98° C. for 30 seconds; 14 cycles of 98° C. for 10 seconds, 65° C. for 30 seconds, 72° C. for 30 seconds; and 72° C. for 5 min. To purify PCR products, 210 μl PCR mixture (from 7 PCR reactions) was mixed with 420 μl NT buffer from NucleoSpin Extract II kit and purified as described above. The final library DNA was eluted with 30 μl of 70° C. elution buffer and DNA concentration was estimated by OD260 measurement.

### REFERENCES

The disclosure of each reference cited is expressly incorporated herein.
1. Ries L A G, S. M., Gurney J G, Linet M, Tamra T, Young J L, Bunin G R Cancer Incidence and Survival among Children and Adolescents: United States SEER Program 1975-1995. in NIH Publication Vol. No. 99-4649 (National Cancer Institute, SEER Program, Bethesda, Md., 1999).
2. Maris, J. M. Recent advances in neuroblastoma. N Engl J Med 362, 2202-11 (2010).
3. Maris, J. M., Hogarty, M. D., Bagatell, R. & Cohn, S. L. Neuroblastoma. Lancet 369, 2106-20 (2007).
4. Capasso, M. & Diskin, S. J. Genetics and genomics of neuroblastoma. Cancer Treat Res 155, 65-84 (2010).
5. Mueller, S. & Matthay, K. K. Neuroblastoma: biology and staging. Curr Oncol Rep 11, 431-8 (2009).
6. Schwab, M. et al. Amplified DNA with limited homology to myc cellular oncogene is shared by human neuroblastoma cell lines and a neuroblastoma tumour. Nature 305, 245-8 (1983).
7. Brodeur, G. M. & Seeger, R. C. Gene amplification in human neuroblastomas: basic mechanisms and clinical implications. Cancer Genet Cytogenet 19, 101-11 (1986).
8. Chen, Y. et al. Oncogenic mutations of ALK kinase in neuroblastoma. Nature 455, 971-4 (2008).
9. George, R. E. et al. Activating mutations in ALK provide a therapeutic target in neuroblastoma. Nature 455, 975-8 (2008).
10. Janoueix-Lerosey, I. et al. Somatic and germline activating mutations of the ALK kinase receptor in neuroblastoma. Nature 455, 967-70 (2008).
11. Mosse, Y. P. et al. Identification of ALK as a major familial neuroblastoma predisposition gene. Nature 455, 930-5 (2008).
12. Cheung, N. K. et al. Association of age at diagnosis and genetic mutations in patients with neuroblastoma. JAMA 307, 1062-71 (2012).
13. Parsons, D. W. et al. The genetic landscape of the childhood cancer medulloblastoma. Science 331, 435-9 (2011).
14. Jones, S. et al. Core signaling pathways in human pancreatic cancers revealed by global genomic analyses. Science 321, 1801-6 (2008).
15. Sjoblom, T. et al. The consensus coding sequences of human breast and colorectal cancers. Science 314, 268-74 (2006).
16. Molenaar, J. J. et al. Sequencing of neuroblastoma identifies chromothripsis and defects in neuritogenesis genes. Nature 483, 589-93 (2012).
17. Clark, M. J. et al. Performance comparison of exome DNA sequencing technologies. Nat Biotechnol (2011).
18. Viswanathan, S. R. et al. Lin28 promotes transformation and is associated with advanced human malignancies. Nat Genet 41, 843-8 (2009).
19. Cotterman, R. & Knoepfler, P. S. N-Myc regulates expression of pluripotency genes in neuroblastoma including lif, klf2, klf4, and lin28b. PLoS One 4, e5799 (2009).
20. Rikova, K. et al. Global survey of phosphotyrosine signaling identifies oncogenic kinases in lung cancer. Cell 131, 1190-203 (2007).
21. Soda, M. et al. Identification of the transforming EML4-ALK fusion gene in non-small-cell lung cancer. Nature 448, 561-6 (2007).
22. Hogarty, M. D. et al. ODC1 is a critical determinant of MYCN oncogenesis and a therapeutic target in neuroblastoma. Cancer Res 68, 9735-45 (2008).
23. Wang, X. et al. Two related ARID family proteins are alternative subunits of human SWI/SNF complexes. Biochem J 383, 319-25 (2004).
24. Fujimoto, A. et al. Whole-genome sequencing of liver cancers identifies etiological influences on mutation patterns and recurrent mutations in chromatin regulators. Nat Genet (2012).
25. Stephens, P. J. et al. The landscape of cancer genes and mutational processes in breast cancer. Nature 486, 400-4 (2012).
26. Jones, S. et al. Frequent mutations of chromatin remodeling gene ARID1A in ovarian clear cell carcinoma. Science 330, 228-31 (2010).
27. Jones, D. T. et al. Dissecting the genomic complexity underlying medulloblastoma. Nature 488, 100-5 (2012).
28. Pugh, T. J. et al. Medulloblastoma exome sequencing uncovers subtype-specific somatic mutations. Nature 488, 106-10 (2012).
29. Versteege, I. et al. Truncating mutations of hSNF5/INI1 in aggressive paediatric cancer. Nature 394, 203-6 (1998).
30. Varela, I. et al. Exome sequencing identifies frequent mutation of the SWI/SNF complex gene PBRM1 in renal carcinoma. Nature 469, 539-42 (2011).
31. Gui, Y. et al. Frequent mutations of chromatin remodeling genes in transitional cell carcinoma of the bladder. Nat Genet 43, 875-8 (2011).
32. Pasqualucci, L. et al. Inactivating mutations of acetyltransferase genes in B-cell lymphoma. Nature 471, 189-95 (2011).
33. Jiao, Y. et al. DAXX/ATRX, MEN1, and mTOR pathway genes are frequently altered in pancreatic neuroendocrine tumors. Science 331, 1199-203 (2011).
34. Schwartzentruber, J. et al. Driver mutations in histone H3.3 and chromatin remodelling genes in paediatric glioblastoma. Nature 482, 226-31 (2012).
35. Wu, G. et al. Somatic histone H3 alterations in pediatric diffuse intrinsic pontine gliomas and non-brainstem glioblastomas. Nat Genet 44, 251-3 (2012).
36. Robinson, G. et al. Novel mutations target distinct subgroups of medulloblastoma. Nature 488, 43-8 (2012).
37. Leary, R. J. et al. Development of personalized tumor biomarkers using massively parallel sequencing. Sci Transl Med 2, 20ra14 (2010).
38. Combaret, V. et al. Circulating MYCN DNA as a tumor-specific marker in neuroblastoma patients. Cancer Res 62, 3646-8 (2002).
39. Yu, A. L. et al. Anti-GD2 antibody with GM-CSF, interleukin-2, and isotretinoin for neuroblastoma. N Engl J Med 363, 1324-34 (2010).
40. Diehl, F. et al. Circulating mutant DNA to assess tumor dynamics. Nat Med 14, 985-90 (2008).
41. Ho, L. & Crabtree, G. R. Chromatin remodelling during development. Nature 463, 474-84 (2010).
42. Ichimura, K. et al. Small regions of overlapping deletions on 6q26 in human astrocytic tumours identified using chromosome 6 tile path array-CGH. Oncogene 25, 1261-71 (2006).
43. Capasso, M. et al. Common variations in BARD1 influence susceptibility to high-risk neuroblastoma. Nat Genet 41, 718-23 (2009).
44. Wang, K. et al. Integrative genomics identifies LMO1 as a neuroblastoma oncogene. Nature 469, 216-20 (2011).
45. Knudson, A. G., Jr., Meadows, A. T., Nichols, W. W. & Hill, R. Chromosomal deletion and retinoblastoma. N Engl J Med 295, 1120-3 (1976).
46. Wu, J. et al. Whole-exome sequencing of neoplastic cysts of the pancreas reveals recurrent mutations in components of ubiquitin-dependent pathways. Proc Natl Acad Sci USA 108, 21188-93 (2011).
47. Bettegowda, C. et al. Mutations in CIC and FUBP1 Contribute to Human Oligodendroglioma. Science 333, 1453-1455 (2011).
48. Wu, J. et al. Recurrent GNAS mutations define an unexpected pathway for pancreatic cyst development. Sci Transl Med 3, 92ra66 (2011).
49. Agrawal, N. et al. Exome sequencing of head and neck squamous cell carcinoma reveals inactivating mutations in NOTCH1. Science 333, 1154-7 (2011).
50. A map of human genome variation from population-scale sequencing. Nature 467, 1061-73 (2010).
51. Kent, W. J. BLAT—the BLAST-like alignment tool. Genome Res 12, 656-64 (2002).
52. Benjamini, Y. & Hochberg, Y. Controlling the False Discovery Rate—a Practical and Powerful Approach to Multiple Testing. Journal of the Royal Statistical Society Series B-Methodological 57, 289-300 (1995).

1. A method to test an individual who has or is suspected of having neuroblastoma, comprising:
testing a biological sample of the individual to detect a deletion or mutation in ARID1B;
detecting the deletion or mutation in the biological sample.

2. (canceled)

3. The method of claim 1 wherein the individual is a pediatric patient.

4. (canceled)

5. (canceled)

6. The method of claim 1 further comprising: repeating the steps of testing and identifying at one or more time points to assess an increase, a decrease, or stability of disease in the individual.

7. The method of claim 6 further comprising: administering a therapy between a first and a second time point and assessing effect of the therapy on the neuroblastoma.

8. The method of claim 1 further comprising: using a primer or probe which specifically detects the deletion or mutation identified in the individual to monitor disease progress in the individual.

9. The method of claim 1 wherein the deletion or mutation affects the A/T-rich interactive domain of ARID1B.

10. The method of claim 1 wherein a deletion is detected.

11. The method of claim 1 wherein a mutation is detected.

12. The method of claim 1 wherein the mutation is a splice site mutation.

13. The method of claim 1 wherein the mutation is a S1436L missense mutation.

14. The method of claim 1 wherein a deletion is identified affecting any one or more of exons 1, 2, 3, 4, 5, 6, 7, 8, or 9.

15. The method of claim 1 further comprising the step of isolating the biological sample from the individual prior to the step of testing.

16. The method of claim 1 wherein the biological sample is selected from the group consisting of blood, serum, urine, sputum, lymph, stool, and tissue.

17. The method of claim 1 further comprising administering an anti-neuroblastoma therapy to the individual.

18. The method of claim 16 wherein cells or shed nucleic acids are collected from the biological sample for use in the step of testing.

19. (canceled)

20. (canceled)

21. (canceled)

22. (canceled)

23. (canceled)

24. The method of claim 1 wherein the step of testing is performed on shed nucleic acids or cells in blood.

25. (canceled)

26. The method of claim 1 wherein the step of testing employs whole-genome, targeted, or exome sequencing.

27. (canceled)

28. (canceled)

29. A method of inhibiting growth of neuroblastoma cells, comprising:
administering to the neuroblastoma cells a polynucleotide encoding a wild-type ARID1B protein, whereby growth of the neuroblastoma cells is inhibited.

30. The method of claim 18 wherein the cells are in culture.

31. The method of claim 18 wherein the cells are in a neuroblastoma model.

32. The method of claim 18 wherein the cells are in a patient's body.

33. The method of claim 18 wherein the cells comprise at least one mutant allele of ARID1B.

34. A method to generate a model of neuroblastoma, comprising:
introducing a mutation into at least one ARID1B allele in a cell, thereby forming a model of neuroblastoma.

35. The method of claim 34 wherein the mutation is introduced into two ARID1B alleles of the cell.

36. A method of testing candidate therapeutic agents for treating neuroblastoma, comprising:
contacting a candidate therapeutic agent with a cell comprising at least one mutant or deleted ARID1B allele, and
measuring the effect of the agent on growth of the cell, wherein an agent which reduces the growth rate of the cell is a more likely candidate therapeutic agent.

37. A method of testing candidate therapeutic agents for treating neuroblastoma, comprising:
contacting an ARID1B protein with an inhibitor;
contacting the ARID1B protein with a candidate therapeutic agent.

38. The method of claim 37 wherein the ARID1B protein is in a cell.

39. The method of claim 27 wherein the inhibitor is an antibody which specifically binds to ARID1B protein.
